(19) United States
(12) Patent Application Publication
(10) Pub. No.: US 2011/0224479 A1
(43) Pub. Date: Sep. 15, 2011

(54) EDDY CURRENT INDUCED HYPERTHERMIA USING CONDUCTIVE PARTICLES

(75) Inventor: Thomas A. Yager, Encinitas, CA (US)

(73) Assignee: Empire Technology Development, LLC, Wilmington, DE (US)

(21) Appl. No.: 12/722,159

(22) Filed: Mar. 11, 2010

(51) Int. Cl. A61N 2/00

(52) U.S. Cl. 600/10

(57) ABSTRACT

Technologies are generally described for hyperthermia based treatment of diseased tissues using conductive particles. Conductive particles of known composition and size distribution may be implanted in diseased tissue and exposed to an alternating magnetic field, which may be tuned to the size of the metal particles to induce eddy currents producing heat in the implanted particles. As the temperature of the metal particles increases, their resistance also increases due to their positive temperature coefficient of resistivity. An antenna placed externally to the body near metal particles may be part of a tuned RF circuit and scanned for resonance. The change either in resonance frequency or circuit impedance may provide tuned feedback, which may be used to control the hyperthermia treatment.

### BACKGROUND

Unless otherwise indicated herein, the materials described in this section are not prior art to the claims in this application and are not admitted to be prior art by inclusion in this section.

Traditional treatments for tumors such as cancer tumors include surgery, chemotherapy, radiotherapy, and combinations of those. While each of these therapy methods is effective in treating certain forms of cancer, other forms may be resistant to their effects. Moreover, side effects of varying degrees are expected with each therapy form. Targeted therapies are a recent development, which aim specific tissues through medication or other methods such as proton radiation or electromagnetically induced heat (hyperthermia). These therapies may reduce side effects while focusing on the diseased tissue.

Induced hyperthermia (elevated temperatures) is one of the targeted therapies for treating diseases such as cancers, heart arrhythmia, and similar ones. Temperatures above ˜41° C. cause necrosis of tumor tissue, while normal tissue is not destroyed until ˜48° C. Thus, diseased tissue portions may be selectively killed allowing healthy tissue to survive the disease. A number of techniques may be used to induce hyperthermia (e.g. in tumors) including Radio Frequency “RF” ablation, microwave ablation, mm-wave ablation, and high intensity focused ultrasound ablation. These techniques provide a means of heating diseased tissue.

The present disclosure recognizes that there are many challenges in controlling temperature during a medical procedure such as induced hypothermia. During surgery, the temperature of the diseased tissue as it is being heated can be monitored using properly positioned temperature probes. However, if induced hyperthermia is used without surgery or post surgery, accurate temperature control through external means may be challenging.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 illustrates use of Eddy current induction system that utilizes conductive particles to induce hyperthermia in a patient for therapeutic purposes;

FIG. 2 illustrates an example Eddy current based controlled hyperthermia system;

FIG. 3 illustrates example Eddy current induction under different temperatures;

FIG. 4 illustrates example resonance modules for measuring temperature in an example controlled hypothermia system;

FIG. 5 illustrates a general purpose computing device, which may be adapted to control an example hyperthermia induction system;

FIG. 6 illustrates a networked environment, where a system for controlled hyperthermia may be implemented;

FIG. 7 is a flow diagram illustrating an example method to implement controlled hyperthermia for therapeutic purposes; and

FIG. 8 illustrates a block diagram of an example computer program product for performing an example method through a computing device;

all arranged in accordance with at least some embodiments of the present disclosure.

### DETAILED DESCRIPTION

In the following detailed description, reference is made to the accompanying drawings, which form a part hereof In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

This disclosure is generally drawn, inter alia, to methods, apparatus, systems, devices, and/or computer program products related to use of conductive particles in hyperthermia treatment of diseased tissues through inducement of Eddy currents.

Briefly stated, technologies are generally described for hyperthermia based treatment of diseased tissues using conductive particles. Conductive particles of known composition and size distribution may be implanted in diseased tissue and exposed to an alternating magnetic field, which may be tuned to the size of the metal particles to induce eddy currents producing heat in the implanted particles. As the temperature of the metal

particles increases, their resistance also increases due to their positive temperature coefficient of resistivity and skin depth effect in the particles. An antenna placed externally to the body near metal particles may be part of a tuned RF circuit and scanned for resonance. The change either in resonance frequency or circuit impedance may provide tuned feedback, which may be used to control the hyperthermia treatment.

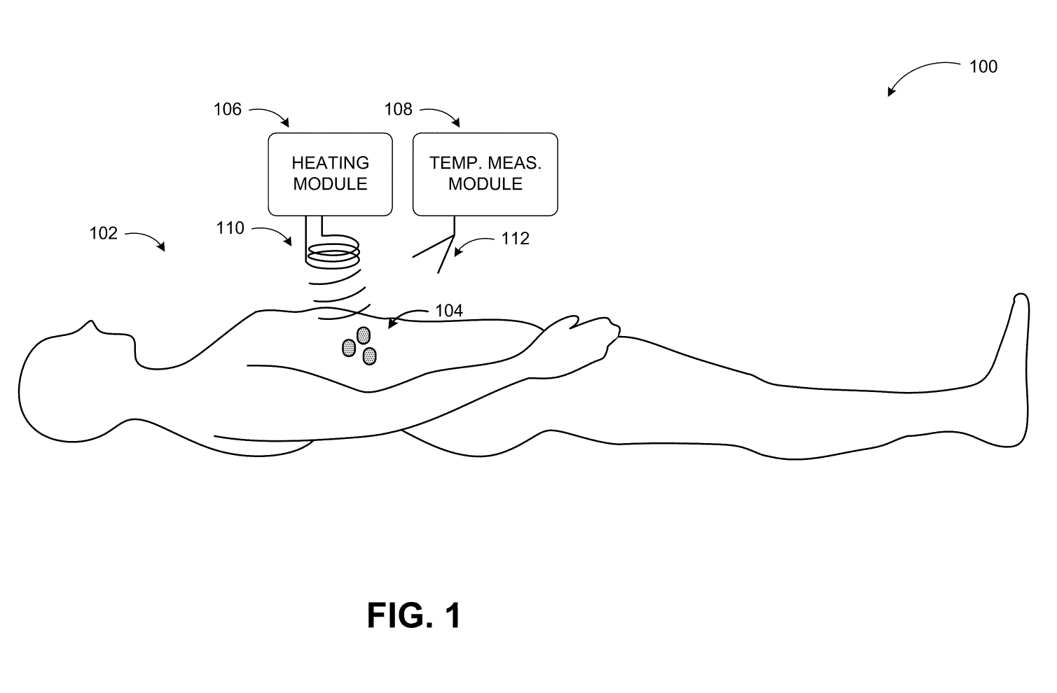

FIG. 1 illustrates use of Eddy current induction system that utilizes conductive particles to induce hyperthermia in a patient for therapeutic purposes according to at least some embodiments described herein.

Localized hyperthermia may be used to destroy diseased tissue in treating a number of illnesses. A number of techniques may be used to deliver heat to the desired location. Examples include focused ultrasound, microwave heating, induction heating, magnetic hyperthermia, or direct application of heat by heated saline solution pumped through catheters. One of the challenges in hyperthermia therapy is delivering the appropriate amount of heat to the correct part of the patient's body. Precise positioning of heat delivery devices such as catheters, microwave or ultrasound applicators, and the like using ultrasound or magnetic resonance imaging are some of approaches.

One example approach involves induction of hyperthermia through insertion of ferromagnetic particles near a tumor. Each particle may comprise at least one ferromagnetic domain. In the presence of an external magnetic field, the magnetic moment of each domain may align to the magnetic field. When the magnetic field switches direction, the magnetic moment may align to the new orientation. Each domain may have a set of preferred orientations of its magnetic moment and energy is required to switch from one orientation to another. As a result, a hysteresis forms and energy is lost in switching from one orientation to another. Thus, heat is delivered to the surrounding tissue. If the particle used for magnetism induced hyperthermia is conductive, Eddy currents may also be induced. The energy loss from the magnetic orientation hysteresis and the Eddy currents results heating of the particles. In an example implementation, alternating current flowing through coils of wire around the body or near the body may produce the alternating magnetic fields.

Temperature control is an important aspect of induced hyperthermia. Therefore, the particles may need to be monitored to determine their temperature. Thus, an example system for inducing hyperthermia based on Eddy currents according to some of the embodiments may be composed of three components (e.g., see FIG. 1): electrically conductive particles 104 implanted into body 102 (into or near the target tissue), a heating module 106 for inducing the Eddy currents in the conductive particles 104, and a temperature measurement module 108 for monitoring the induced temperature in the body 102. Heating module 106 may be configured to generate an electromagnetic field through an antenna 110 to induce Eddy currents in the conductive particles 104, which in turn generates heat used for hyperthermia treatment. According to some embodiments, the particles may be non-ferromagnetic and rely on the Eddy currents alone to generate heat for hyperthermia. According to other embodiments, the heat used in the hypothermia treatment may be generated by a combination of Eddy currents and alternating magnetic orientations creating additional heat with the ferromagnetic particles.

Since accurate temperature measurement through non-invasive methods is a challenge (especially when small target areas are being used), the temperature of the heating particles may be determined through a resonant circuit, which is partially formed by the particles. According to at least some embodiments described herein, temperature measurement module 108 may include a resonant circuit and an antenna 112 located in a vicinity of the conductive particles 104 and oriented such that an effective inductance associated with the resonant circuit is influenced by the Eddy currents flowing in the particles. A resonant frequency associated with the resonant circuit may be sensitive to the temperature of the particles. Furthermore, an effective impedance of the resonant circuit near resonance may be decreased due to energy lost to the particles.

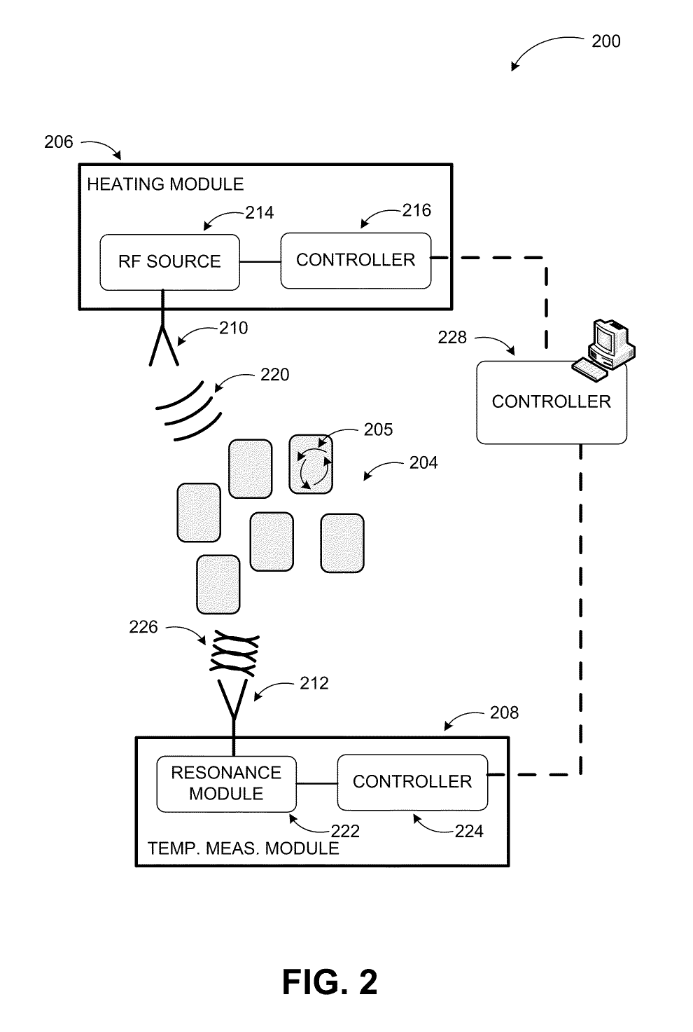

FIG. 2 illustrates an example Eddy current based controlled hyperthermia system 200 that is arranged in accordance with at least some embodiments described herein. Example system 200 may include conductive particles 204, a heating module 206, and a temperature measurement module 208. The heating module 206 and the temperature measurement module 208 may be controlled by a controller device 228, which may be external (e.g., remote) from system 200.

The heating module 206 and the temperature module 208 may each include their own controllers 216 and 224, respectively. Controller 216 may be configured to manage an RF source 214 to generate an electromagnetic wave 220 with a predefined frequency that may be transmitted to conductive particles 204 through antenna 210. Conductive particles 204 may be ferromagnetic particles that can generate heat as a result of the alternating magnetic orientation as well as from the flow of Eddy currents 212 in the particles. According to some embodiments, the particles may be non-ferromagnetic particles and the heat generated by these particles result from Eddy currents, which are induced by the electromagnetic field 220.

The temperature of the conductive particles 204 may be measured by resonance module 222 based on its interaction (226) with the particles through antenna 212. The particles may form a part of the resonant circuit and a change in either the resonant frequency or effective impedance of the circuit may provide tuned feedback for determining the temperature. The particles change their inductance, L, by producing a self inductance in the circuit. This self inductance is a function of the amount of eddy currents in the particles. A resonant frequency of the circuit may be expressed as:

ωₒ=1/\sqrt{LC} [1]

Typically, impedance is due to actual components such as resistors, capacitors, inductors, etc. However, effective impedance is usually referred to when a combination of circuit components, plus other sources such as parasitic effects from circuits and circuit boards, the effective of air and temperature, skin, etc. are referred to. The temperature information from controller 224 may be used by controller device 228 to adjust a level, duration, and/or frequency of the electromagnetic field 220 through controller 216 such that the hyperthermia treatment can be effectively administered.

Controller device 228 may be a general purpose computing device or a special purpose computing device that may be comprised as a standalone computer, a networked computer system, a general purpose processing unit (e.g., a micro-processor, a micro-controller, a digital signal processor or DSP, etc.), a special purpose processing unit (e.g., a specialized controller, an application specific integrated circuit or ASIC) or some other similarly configured devices. Controller device 228 may be adapted to control an initial frequency and/or level of the electromagnetic field 220 as well as subsequent adjustments that may be made responsive to the measured temperature of the conductive particles 204. Controller device 228 may further be configured to generate records of the treatment (e.g., data logging) and adjust positions and/or orientations of the RF source 214, resonance module 222, and their respective antennas.

Some example ferromagnetic particles are transition metal oxides. The magnetization of ferromagnetic particles may vary with temperature according to Bloch's Law, which defines the temperature dependence of the magnetization for ferromagnetic or ferromagnetic materials as:

M(T)=M(0)*(1−(T/T_{C})^{3/2}) [2]

where T_{C} is the Curie temperature. At temperatures above T_{C} a material is paramagnetic. At temperatures below T_{C}, magnetization is spontaneous. The variation of magnetization with temperature is significant only near the Curie temperature (T_{C}) of the material. For transition metal oxides, T_{C} is typically a temperature that is greater than about 500° C. to about 600° C.

Ferromagnetic materials with lower Curie temperatures tend to be more toxic and unstable. Therefore, it may be difficult to measure any changes in ferromagnetic properties over the range of about 35° C. to about 50° C. Non-ferromagnetic metal particles may be unsuitable for magnetism induced hyperthermia, since their absorption of energy is less than in ferromagnetic particles. However, Eddy currents may be induced into non-ferromagnetic conductive particles to produce heat, and the temperature coefficient of resistivity may be used for temperature control.

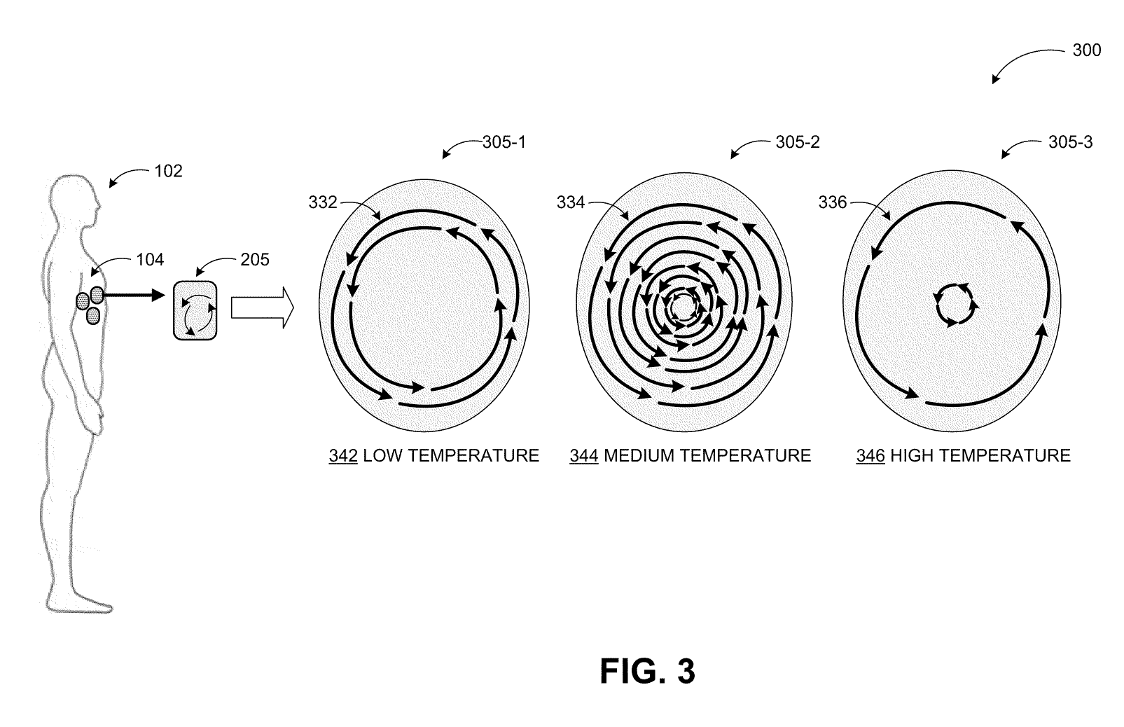

FIG. 3 illustrates example Eddy current induction under different temperatures, in accordance with at least some embodiments of the present disclosure. Diagram 300 shows how different Eddy currents may be induced in conductive particles such as one of the (205) conductive particles 104 implanted in human body 102 (as in FIG. 1) for generating hyperthermia. Eddy currents are closed loops of induced current circulating in planes perpendicular to the magnetic flux. The Eddy currents normally travel parallel to an excitation coil's winding (e.g. antenna 212 of FIG. 2), and current flow is limited to the area of the inducing magnetic field. The skin effect within the particles may have a large influence on the amount of eddy currents flowing within the particle.

The skin effect arises when the Eddy currents flowing in a metallic object at any depth produce magnetic fields which oppose the primary field, thus reducing the net magnetic flux and causing a decrease in current flow as the depth increases. Alternatively, Eddy currents near the surface may be viewed as shielding the coil's magnetic field, thereby weakening the magnetic field at greater depths and reducing induced currents.

Conductive particle 305-1 is an example of Eddy currents at low temperature (342). The currents 332 tend to concentrate near the surface of the conductive particle 305-1 without penetrating the central regions of the particle. At medium temperature 344, Eddy currents 334 may be found throughout the conductive particle 305-2. On the other hand, if the temperature reaches high values (346), the current density may decrease as shown in conductive particle 305-3. Eddy currents 336 are distributed throughout the particle, but much less dense than the optimum temperature distribution shown in conductive particle 305-2.

The temperature ranges for different Eddy current distributions as shown in diagram 300 are relative and depend on parameters such as a composition of the conductive particles, a size of the conductive particles, a frequency of excitation signal, and similar ones. Thus, a particle size and composition, as well as a frequency of excitation may be selected to induce desired temperature increase in the conductive particles.

The skin depth for Eddy currents is defined as:

$$δ = \sqrt{\frac{2}{{ωσμ}_{0} {μ}_{r}}} \qquad [ 2 ]$$

where δ is the skin depth, ω is the angular frequency of the magnetic field, σ is the electrical conductivity, μ₀ is the absolute magnetic permeability, and μᵣ is the relative magnetic permeability of the material compared to air. As a metal particle is heated, electrical conductivity (σ) decreases and skin depth (δ) increases due to the positive temperature coefficient of resistivity of metals. Thus, in a hyperthermia induction system according to some of the embodiments described herein, the particles and the magnetic frequency may be chosen such that the skin depth is less than a radius of the particles. For example, if platinum particles and an excitation frequency of about 2.5 GHz is used, the skin depth is about 3.2 μm. Thus, in this example, a particle diameter of greater than about 6.4 μm may be used for effective Eddy current based heating.

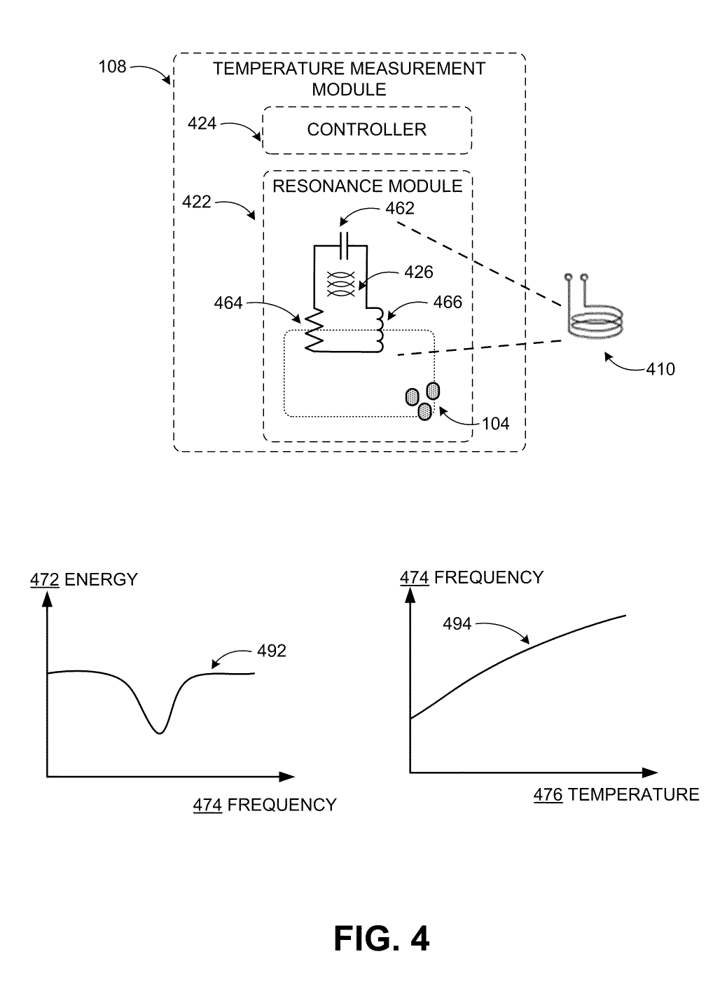

FIG. 4 illustrates an example resonance module for measuring temperature in an example controlled hypothermia system that is arranged according to at least some embodiments described herein.

Temperature measurement module 108 (as in FIG. 1) may include a controller 424 and resonance module 422. In measuring the temperature of the particles, a number of interactions may be considered. For example, due to the positive temperature coefficient of the metal particles, an increase in temperature increases their resistivity (e.g. ˜0.39%/° C.) for platinum. An increase in resistivity increases the skin depth of induced Eddy currents. If the particle radius is at or below the skin depth at body temperature, an increase in temperature may decrease the Eddy currents due to both the change in resistivity and less material for the Eddy currents to flow.

With an antenna 410 of an RF resonance circuit in the vicinity of the particles, the inductance of the circuit may be influenced by the Eddy currents in the particles. As a result, the resonant frequency of the circuit becomes sensitive to the temperature of the metal particles. In addition, the impedance of the circuit near resonance may be increase relative to the expected impedance due to energy that may be lost to the particles.

Thus, resonance module 460 may be modeled as a basic resonant circuit with a capacitive element 462, an inductive element 466, and a resistive element 464. Conductive particles 104 form part of the resonant circuit by RF interaction 426 through an antenna (410) of the resonance module 460 placed near the conductive particles 104. As discussed above, the inductance of the circuit is influenced by the Eddy currents, which results in the resonant frequency being dependent on the temperature of the particles in addition to the increase of the impedance near resonance as shown by graphs 492 and 494, which illustrate a change of energy 472 in the circuit with frequency 474 and a change of temperature 476 with frequency 474.

While embodiments have been discussed above using specific examples, components, and configurations, they are intended to provide a general guideline to be used for inducing controlled hyperthermia through Eddy currents in conductive particles. These examples do not constitute a limitation on the embodiments, which may be implemented using other components, current induction or temperature measurement schemes, and/or configurations using the principles described herein. For example, a number of antenna types, positions, and/or particle types may be used in other embodiments. Control of parameters such as RF field levels, durations of RF field, positions of antenna(s), etc. may be implemented through specific algorithms executed by one or more computing devices or controllers.

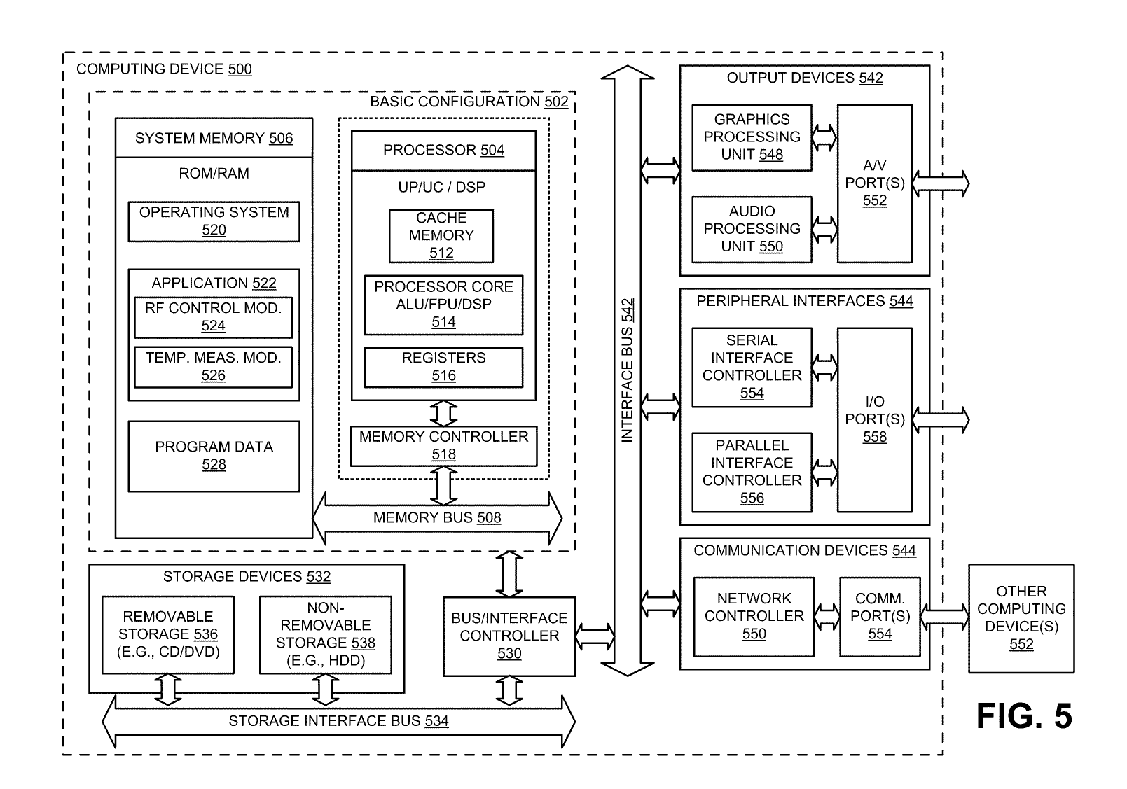

I guess this is necessary. FIG. 5 illustrates a general purpose computing device 500, which may be adapted to control an example hyperthermia induction system that is arranged according to at least some embodiments of the present disclosure. In a very basic configuration 502, computing device 500 typically includes one or more processors 504 and a system memory 506. A memory bus 508 may be used for communicating between processor 504 and system memory 506.

Depending on the desired configuration, processor 504 may be of any type including but not limited to a microprocessor (μP), a microcontroller (μC), a digital signal processor (DSP), or any combination thereof Processor 504 may include one more levels of caching, such as a level cache memory 512, a processor core 514, and registers 516. Example processor core 514 may include an arithmetic logic unit (ALU), a floating point unit (FPU), a digital signal processing core (DSP Core), or any combination thereof An example memory controller 518 may also be used with processor 504, or in some implementations memory controller 518 may be an internal part of processor 504.

Depending on the desired configuration, system memory 506 may be of any type including but not limited to volatile memory (such as RAM), non-volatile memory (such as ROM, flash memory, etc.) or any combination thereof System memory 506 may include an operating system 520, one or more applications 522, and program data 528. Application 522 may include an RF control module 524 that is arranged to adjust operational parameters of an RF source for inducing Eddy currents in implanted conductive particles as discussed above. Application 522 may further include a temperature measurement module 526 that is arranged to determine a temperature of the particles through a resonant circuit using a resonant frequency and/or impedance of the circuit. Program data 528 may include any data associated with controlling the RF source and measuring the temperature of the conductive particles as discussed above (e.g., FIGS. 3 and 4). In some embodiments, application 522 may be arranged to operate with program data 528 on operating system 520 such that Eddy current induced hyperthermia may be controlled as described herein. This described basic configuration 502 is illustrated in FIG. 5 by those components within the inner dashed line.

Computing device 500 may have additional features or functionality, and additional interfaces to facilitate communications between basic configuration 502 and any required devices and interfaces. For example, a bus/interface controller 530 may be used to facilitate communications between basic configuration 502 and one or more data storage devices 532 via a storage interface bus 534. Data storage devices 532 may be removable storage devices 536, non-removable storage devices 538, or a combination thereof Examples of removable storage and non-removable storage devices include magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include volatile and nonvolatile, removable and non-removable media implemented in any method or technology for storage of information, such as computer readable instructions, data structures, program modules, or other data.

System memory 506, removable storage devices 536 and non-removable storage devices 538 are examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 500. Any such computer storage media may be part of computing device 500.

Computing device 500 may also include an interface bus 540 for facilitating communication from various interface devices (e.g., output devices 542, peripheral interfaces 544, and communication devices 546) to basic configuration 502 via bus/interface controller 530. Example output devices 542 include a graphics processing unit 548 and an audio processing unit 550, which may be configured to communicate to various external devices such as a display or speakers via one or more A/V ports 552. Example peripheral interfaces 544 include a serial interface controller 554 or a parallel interface controller 556, which may be configured to communicate with external devices such as input devices (e.g., keyboard, mouse, pen, voice input device, touch input device, etc.) or other peripheral devices (e.g., printer, scanner, etc.) via one or more I/O ports 558. An example communication device 546 includes a network controller 560, which may be arranged to facilitate communications with one or more other computing devices 562 over a network communication link via one or more communication ports 564.

The network communication link may be one example of a communication media. Communication media may typically be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) and other wireless media. The term computer readable media as used herein may include both storage media and communication media.

Computing device 500 may be implemented as a portion of a small-form factor portable (or mobile) electronic device such as a cell phone, a personal data assistant (PDA), a personal media player device, a wireless web-watch device, a personal headset device, an application specific device, or a hybrid device that include any of the above functions. Computing device 500 may also be implemented as a personal computer including both laptop computer and non-laptop computer configurations. Moreover computing device 500 may be implemented as a networked system or as part of a general purpose or specialized server.

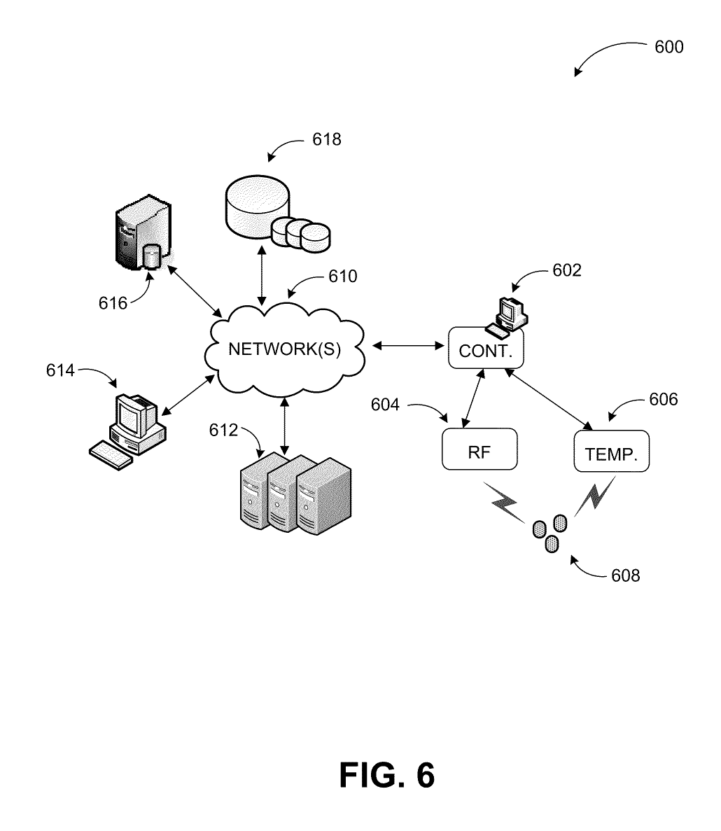

FIG. 6 illustrates a networked environment, where a system for controlled hyperthermia may be implemented in accordance with at least some embodiments described herein. A control system managing Eddy current induced hyperthermia may be implemented through separate applications, one or more integrated applications, one or more centralized services, or one or more distributed services on one more computing devices. Diagram 600 illustrates an example of a distributed system implementation through networks 610.

As discussed previously, induction of Eddy currents (and/or magnetic orientation alternation) may be controlled by a local controller 604. The temperature may be measured through measurement module 606. A controller (e.g. a general purpose computing device) 602 may be configured to collect temperature data, provide feedback to the RF source controller 602, and/or provide feedback information to an application or service executed on computing device 614 or one or more of the servers 612 through network(s) 610. The application or service may be adapted to manage one or more hyperthermia induction systems, maintain patient data, provide initial configuration information to controller 602, and perform similar tasks. Patient data and other data associated with the operation of hyperthermia induction system may be stored in one or more data stores such as data stores 618 and be directly accessible through network(s) 610. Alternatively, data stores 618 may be managed by a database server 616.

Network(s) 610 may comprise any topology of servers, clients, switches, routers, modems, Internet service providers (ISPs), and any appropriate communication media (e.g., wired or wireless communications). A system according to embodiments may have a static or dynamic network topology. Network(s) 610 may include a secure network such as an enterprise network (e.g., a LAN, WAN, or WLAN), an unsecure network such as a wireless open network (e.g., IEEE 802.11 wireless networks), or a world-wide network such (e.g., the Internet). Network(s) 610 may also comprise a plurality of distinct networks that are adapted to operate together. Network(s) 610 can be configured to provide communication between the nodes described herein. By way of example, and not limitation, network(s) 610 may include wireless media such as acoustic, RF, infrared and other wireless media. Furthermore, network(s) 610 may be portions of the same network or separate networks.

Example embodiments may also include methods. These methods can be implemented in any number of ways, including the structures described herein. One such way is by machine operations, of devices of the type described in the present disclosure. Another optional way is for one or more of the individual operations of the methods to be performed in conjunction with one or more human operators performing some of the operations while other operations are performed by machines (e.g., devices adapted to perform operations). Human operators need not be collocated with each other, but instead can be located about a machine that performs a portion of the overall program or process. In other examples, the human interaction can be automated such as by pre-selected criteria that are machine automated.

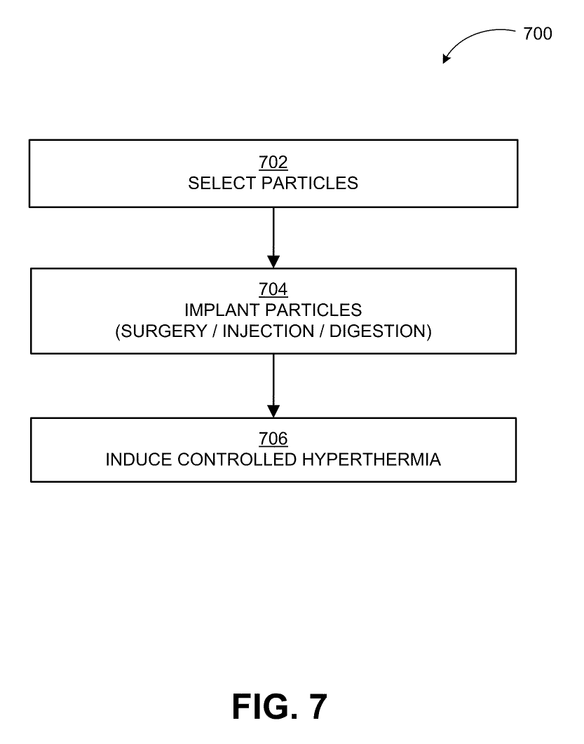

FIG. 7 is a flow diagram illustrating an example method to implement controlled hyperthermia for therapeutic purposes, arranged in accordance with at least some embodiments described herein.

Process 700 for implementing controlled hyperthermia begins with operation 702, “SELECT PARTICLES”. The particles may be selected from a reasonably uniform sized powder of a non-toxic conductor (for intra-body applications such as the one shown in FIG. 1). The material may include, but is not limited to, platinum, gold, or other encapsulated metal particles. According to at least some embodiments, a particle radius near the skin depth for the excitation frequency may be selected. For example, a particle radius near about 3.2 μm may be used for an excitation frequency of about 2.5 GHz for platinum based particles. Other particle compositions, sizes, and frequencies may be used as well. Furthermore, the conductive particles may be encapsulated in insulating materials such as glass, ceramic, polymers, and the like.

Operation 702 may be followed by operation 704, “IMPLANT PARTICLES”, where the particles (e.g. particles 104 if FIG. 1) may be implanted into and nearby the target tissue. The implantation may be by surgical insertion, injection of a colloid that includes the particles, digestion of a particle containing solution, or similar methods. Once the particles are implanted, controlled hyperthermia may be induced through induction of Eddy currents in the particles (optionally supplemented by magnetic orientation alternation) with feedback from temperature measurement as discussed herein at operation 706 “INDUCE CONTROLLED HYPERTHERMIA.” Operation 706 may be performed by a system such as the one shown in diagram 200 of FIG. 2. The Eddy current induced hyperthermia may be applied in place of or in addition to other forms of therapy such as surgery, chemotherapy, and/or other comparable medical procedures.

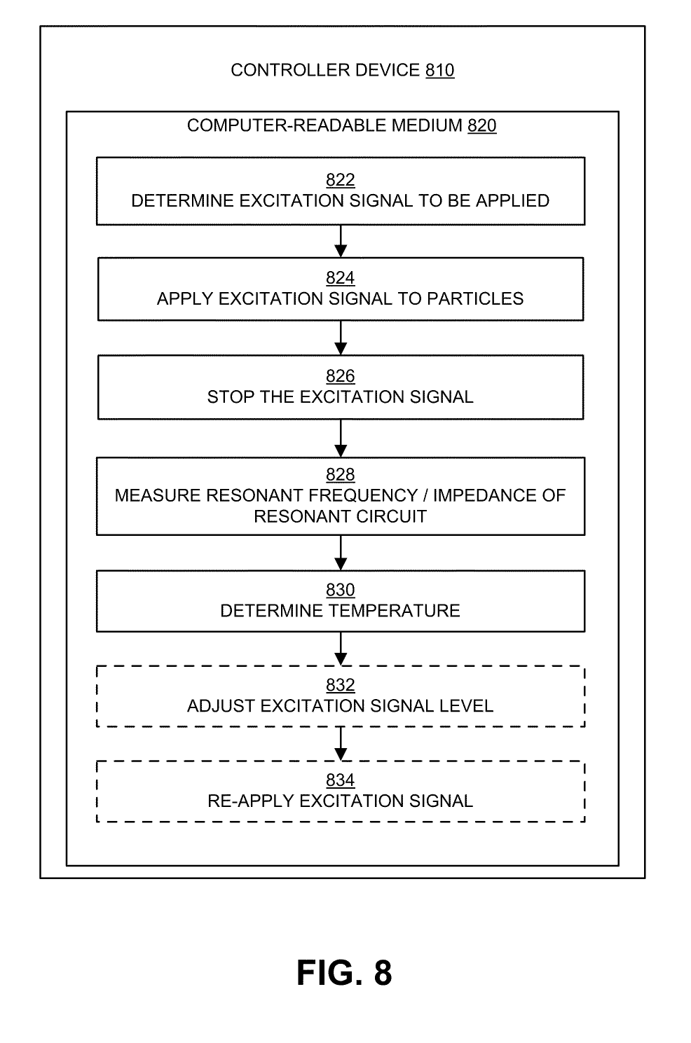

FIG. 8 illustrates a block diagram of an example computer program product for performing an example method through a computing device (e.g., device 500 in FIG. 5), arranged in accordance with at least some embodiments of the present disclosure. In some examples, as shown in FIG. 8, computer readable medium 820 may include machine readable instructions that, when executed by a computing device (e.g., controller device 810) adapt the computing device to provide at least a portion of the functionality described above with respect to FIG. 1 through FIG. 4. For example, referring to controller device 810, one or more modules of controller device 810 may be configured to undertake one or more of the operations shown in FIG. 8.

A process of controlling Eddy current induced hyperthermia may begin with operation 822, “DETERMINE EXCITATION SIGNAL TO BE APPLIED.” At operation 822, an initial RF excitation signal level and duration may be determined (e.g., by controller device 810) and control parameters can be provided (e.g., by controller device 810) to an RF source.

Operation 822 may be followed by operation 824, “APPLY EXCITATION SIGNAL TO PARTICLES.” At operation 824, the RF source subjects the particles to an alternating electromagnetic field (e.g. electromagnetic field 220 of FIG. 2) inducing Eddy current in the particles 204 and thereby heating the particles. The RF source (e.g., RF source 214 of FIG. 2) can be adapted to apply the excitation signal to the particles via an antenna with an amount (e.g., signal level, etc.) and duration of time responsive to the control parameters determined at operation 822 via various control signals that may be provided from the controller device (e.g., controller 216 or controller 228, or controller 810).

Operation 824 may be followed by operation 826, “STOP THE EXCITATION SIGNAL.” At operation 826, the electromagnetic field may be stopped briefly to allow measurement of the temperature of the particles through a resonance circuit such as resonance module 222 of FIG. 2. In some examples, the interruption of the excitation can be implemented via various control signals that may be provided from the controller device (e.g., controller 216 or controller 228, or controller 810).

Operation 826 may be followed by operation 828, “MEASURE RESONANT FREQUENCY/IMPEDANCE OF RESONANT CIRCUIT.” At operation 828, a resonant frequency of the resonant circuit formed by various components of the system (e.g., resonance module 222, antenna 212, and particles 204) may be determined (e.g., via temperature measurement module 208). As discussed previously, Eddy currents 212 may influence the resonant frequency of the circuit in a temperature dependent manner in addition to the impedance of the circuit changing with the temperature of the particles. Thus, the impedance changes may also be utilized to determine temperature information.

Operation 828 may be followed by operation 830, “DETERMINE TEMPERATURE.” At operation 830, the temperature of the particles may be determined (e.g., via controller device 810, controller 224 or controller 228) based on the measured resonant frequency and/or the impedance of the resonant circuit. According to some embodiments, calibration measurements may be performed by the temperature measurement module prior to actual hyperthermia treatment.

Operation 830 may be followed by optional operation 832, “ADJUST EXCITATION SIGNAL LEVEL.” At operation 832, the level of applied RF signal for inducing Eddy currents may be adjusted based on feedback obtained from the measured temperature. This may be accomplished manually or by an automated process controller such via one or more of controller 228 and/or controller 216 of FIG. 2. In addition to the level of the RF signal, a duration of the signal, a position of the antenna 220, etc. may also be adjusted based on the same feedback.

Optional operation 832 may be followed by optional operation 834, “REAPPLY EXCITATION SIGNAL”, where the excitation signal can be reactivated by the RF source (e.g., RF source 214 of FIG. 2) with the adjusted parameters via various control signals that may be provided from the controller device (e.g., controller 216 or controller 228, or controller 810). As discussed previously, the processors and controllers performing these operations are example illustrations and should not be construed as limitations on embodiments. The operations may also be performed by other computing devices or modules integrated into a single computing device or implemented as separate machines.

The operations discussed above are for illustration purposes. Controlling Eddy current induced hyperthermia may be implemented by similar processes with fewer or additional operations. In some examples, the operations may be performed in a different order. In some other examples, various operations may be eliminated. In still other examples, various operations may be divided into additional operations, or combined together into fewer operations.

There is little distinction left between hardware and software implementations of aspects of systems; the use of hardware or software is generally (but not always, in that in certain contexts the choice between hardware and software may become significant) a design choice representing cost vs. efficiency tradeoffs. There are various vehicles by which processes and/or systems and/or other technologies described herein may be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples may be implemented, individually and/or collectively, by a wide range of hardware, software, firmware, or virtually any combination thereof In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, may be equivalently implemented in integrated circuits, as one or more computer programs running on one or more computers (e.g., as one or more programs running on one or more computer systems), as one or more programs running on one or more processors (e.g. as one or more programs running on one or more microprocessors), as firmware, or as virtually any combination thereof, and that designing the circuitry and/or writing the code for the software and or firmware would be well within the skill of one of skill in the art in light of this disclosure.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein may be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors.

A typical data processing system may be implemented utilizing any suitable commercially available components, such as those typically found in data computing/communication and/or network computing/communication systems. The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermediate components. Likewise, any two components so associated may also be viewed as being “operably connected”, or “operably coupled”, to each other to achieve the desired functionality, and any two components capable of being so associated may also be viewed as being “operably couplable”, to each other to achieve the desired functionality. Specific examples of operably couplable include but are not limited to physically connectable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

Furthermore, in those instances where a convention analogous to “at least one of A, B, and C, etc.” is used, in general such a construction is intended in the sense one having skill in the art would understand the convention (e.g., “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “ a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method for generating hyperthermia through Eddy current induction in implanted conductive particles, the method comprising:
applying an alternating electromagnetic field from a Radio Frequency (RF) source wherein the alternating electromagnetic field is effective to induce Eddy currents in the implanted conductive particles; and
determining an approximate temperature of the implanted conductive particles based on an effect of the Eddy currents on one or more of a resonance frequency and/or effective impedance of a resonant circuit, wherein the resonance circuit effectively includes the implanted conductive particles.

2. The method according to claim 1, further comprising determining a frequency of the alternating electromagnetic field based on one or more of:
a size, an electrical conductivity, and/or a magnetic permeability of the implanted conductive particles.

3. The method according to claim 1, wherein the implanted conductive particles are ferromagnetic and the method further comprises generating a magnetic orientation alternation in the ferromagnetic implanted conductive particles.

4. The method according to claim 1, further comprising adjusting one or more of a position and/or an orientation of one or more antennas in response to the determined temperature, wherein the one or more antennas are coupled to the RF source and wherein the one or more antennas form part of the resonant circuit.

5. The method according to claim 1, further comprising adjusting one or more of a level and a duration of the alternating electromagnetic field in response to the determined temperature.

6. The method according to claim 1, further comprising calibrating one or more of a duration of the alternating electromagnetic field, a level of the alternating electromagnetic field, and/or a position of antenna of the RF source prior to beginning hyperthermia treatment through Eddy current induction.

7. The method according to claim 6, further comprising applying the hyperthermia treatment through Eddy current induction in conjunction with one or more of surgical treatment, chemotherapy, and/or radiotherapy.

8. The method according to claim 1, wherein conductive particles are implanted in or near a target tissue through one or more of surgically inserting, injecting a colloid that includes the conductive particles, and/or causing digestion of a solution that includes the conductive particles in or near diseased tissue.

9. The method according to claim 1, wherein the implanted conductive particles comprise one or more of: platinum, gold, and/or encapsulated metals.

10. The method according to claim 9,wherein the
encapsulated metals are encapsulated with one or more of glass, ceramic, and/or polymers.

11. An apparatus for generating hyperthermia through Eddy current induction in implanted conductive particles, the apparatus comprising:
a Radio Frequency (RF) source device adapted to transmit an alternating electromagnetic field through an antenna to the implanted conductive particles, wherein Eddy currents are induced in the implanted conductive particles in response to the alternating electromagnetic field such that a temperature of the implanted conductive particles is increased to a controlled level;
a controller adapted to determine one or more of an initial level of the alternating electromagnetic field, a duration of the alternating electromagnetic field, and/or a position of the antenna relative to the implanted conductive particles; and
a temperature measurement device adapted to determine an approximate temperature of the implanted conductive particles based on an effect of the Eddy currents on one or more of a resonance frequency and/or effective impedance of a resonant circuit, wherein the resonance circuit effectively includes the implanted conductive particles.

12. The apparatus according to claim 11, wherein the RF source device is further adapted to generate an alternating magnetic field such that ferromagnetic implanted conductive particles are heated based on alternation of their magnetic orientations in addition to the induced Eddy currents.

13. The apparatus according to claim 11, wherein the controller is further adapted to adjust the level of the alternating electromagnetic field, the duration of the alternating electromagnetic field, and/or the position of the antenna relative to the implanted conductive particles in response to the approximate temperature information provided by the temperature measurement device.

14. The apparatus according to claim 11, wherein the controller is further adapted to determine a frequency of the alternating electromagnetic field based on one or more of: a size, an electrical conductivity, and a magnetic permeability of the implanted conductive particles.

15. The apparatus according to claim 14, wherein the size of the implanted conductive particles is selected such that an average radius of the implanted conductive particles is more than a skin depth for the Eddy currents induced in the implanted conductive particles.

16. An apparatus for determining a temperature of implanted conductive particles employed for generating hyperthermia through Eddy current induction, comprising:
an antenna for interacting with the implanted conductive particles, wherein the antenna is effective to form part of a resonant circuit that includes the implanted conductive particles; and
a controller adapted to determine an approximate temperature of the implanted conductive particles based on one or more of a resonant frequency and an effective impedance of the resonant circuit.

17. The apparatus according to claim 16, wherein the controller is further adapted to adjust a position of the antenna based on one or more initial measurements.

18. The apparatus according to claim 16, wherein the controller is further adapted to provide the approximate temperature as feedback to a heating apparatus generating the hyperthermia.

19. The apparatus according to claim 16, wherein the antenna is placed in a vicinity of the implanted conductive particles implanted in or near a target tissue.

20. A system for generating controlled hyperthermia through Eddy current induction in implanted conductive particles, the system comprising:
a heating module adapted to increase a temperature of the implanted conductive particles implanted in or near a target tissue by inducing Eddy currents in the implanted conductive particles through an alternating electromagnetic field generated by a Radio Frequency (RF) source; and
a temperature measurement module adapted to determine an approximate temperature of the implanted conductive particles through a resonance circuit, wherein the resonance circuit effectively includes the implanted conductive particles.

21. The system according to claim 20, further comprising:
a controller coupled to the heating module and the temperature measurement module, wherein the controller is adapted to provide control parameters to the heating module in response to the determined approximate temperature by the temperature measurement module.

22. The system according to claim 21, wherein the controller is one of a standalone computer, a networked computer system, a micro-processor, a micro-controller, a digital signal processor, or a special purpose processing unit.

23. The system according to claim 21, wherein the controller is further adapted to record temperature and applied electromagnetic field information.

24. The system according to claim 20, wherein one or more of a size and a composition of the implanted conductive particle is selected based on one or more of a desired heat to be generated in the target tissue and a frequency of the RF source.

25. The system according to claim 24, wherein the implanted conductive particles are made from ferromagnetic material and the heating module is further adapted to increase the temperature of the implanted conductive particles through magnetic orientation alternation.

26. The system according to claim 20, wherein temperature measurement module is adapted to determine the approximate temperature of the implanted conductive particles based on one or more of an effective impedance and a resonant frequency of the resonance circuit.
